(19) United States
(12) Patent Application Publication
(10) Pub. No.: US 2011/0190778 A1
(43) Pub. Date: Aug. 4, 2011

(54) Multiple Port Introducer for Thrombolysis

(76) Inventors: Paul Arpasi, Beverly Hills, MI (US); Michael Zennedjian, Farmington Hills, MI (US)

(21) Appl. No.: 13/055,744

(22) PCT Filed: Jul. 28, 2009
(86) PCT No.: PCT/US09/51899

§ 371 Date: Apr. 13, 2011

Related U.S. Application Data
(60) Provisional application No. 61/083,979, filed on Jul. 28, 2008.

(51) Int. Cl. A61B 17/00

(52) U.S. Cl. 606/108

(57) ABSTRACT

The present invention provides a multiple port infusion introducer including an introducer sheath having a tubular introducer wall defining an introducer lumen formed therethrough. The introducer wall includes a proximal portion extending to a distal portion along a longitudinal axis. A plurality of ports is positioned proximate the proximal end of the introducer sheath. Each port has a tubular port wall defining a port lumen formed therethrough. Each port lumen is in fluid communication with the introducer lumen. Each port wall has a proximal end extending to a distal end along a central axis. A delivery member is disposed through the proximal end of one of the ports and extends through the port lumen and into the introducer lumen proximate the proximal end of the introducer sheath.

### BACKGROUND

1. Field of the Invention

The present invention generally relates to the field of medical devices, and more particularly to a system and method for inserting multiples catheters for locally delivering fluids or agents within the body of a patient.

2. Description of Related Art

Many different medical device systems and methods have been previously disclosed for locally delivering fluids or other agents into various body regions, including body lumens such as vessels, or other body spaces such as organs or heart chambers. Local “fluid” delivery systems may include drugs or other agents, or may even include locally delivering the body's own fluids, such as artificially enhanced blood transport. Local “agent” delivery systems are herein generally intended to relate to introduction of a foreign composition as an agent into the body, which may include a drug or other useful or active agent, and may be in a fluid form or other form such as gels, solids, powders, gases, etc. It is to be understood that reference to only one of the terms fluid, drug, or agent with respect to local delivery systems may be made variously in this disclosure for illustrative purposes, but is not generally intended to be exclusive or omissive of the others; they are to be considered interchangeable where appropriate according to one of ordinary skill in the art unless specifically described to be otherwise.

Various diagnostic systems and procedures have been developed using local delivery of dye (e.g. radiopaque “contrast” agent) or other diagnostic agents, wherein an external monitoring system is able to gather important physiological information based upon the diagnostic agent's movement or assimilation in the body at the location of delivery and/or at other locations affected by the delivery site. Angiography is one such practice that uses a hollow, tubular angiography catheter for locally injecting radiopaque dye into a blood chamber or vessel, such as for example coronary arteries in the case of coronary angiography, or in a ventricle in the case of cardiac ventriculography.

Other systems and methods have been disclosed for locally delivering therapeutic agents into a particular body tissue within a patient via a body lumen. For example, angiographic catheters of the type just described above, and other similar tubular delivery catheters, have also been disclosed for use in locally injecting treatment agents through their delivery lumens into spaces within the body. More detailed examples of this type include local delivery of thrombolytic drugs such as TPA™, heparin, cumadin, or urokinase into areas of existing clot or thrombogenic implants or vascular injury. However, current procedures requiring multiple catheters for delivering therapeutic agents within a patient use multiple access sites.

Thrombolytic strategies are based on arterial anatomy of a small diameter. Venous thrombolysis is becoming more common in practice, but single lumen coaxial access limits treatment options. Thus, a need exists for improved devices and methods for local delivery of agents into various branch arteries or veins through a single access site. Further, a need exists for a coaxial introducer of variable size with multiple hemostatic access ports for the delivery of multiple thrombolytic catheters, wires, snares or low profile angioplasty balloons from a single access site.

### SUMMARY OF THE INVENTION

In satisfying the above need, as well as overcoming the enumerated drawbacks and other limitations of the related art, at least one embodiment of the present invention provides a multiple port thrombolysis infusion introducer having multiple hemostatic access ports capable of simultaneously placing multiple thrombolytic catheters, wires, snares or low profile angioplasty balloons within a patient for the delivery of drugs and therapeutic agents through a single access site. Embodiments of the multiple port introducer of the present invention allow multiple concurrent selective thrombolyses, increased surface area of drug delivery within large caliber vessels, complex foreign body retrieval, and multiple low profile angioplasties in patients where multiple access points are undesired or unavailable.

In a first embodiment of the present invention, a multiple port infusion introducer includes an introducer sheath having a tubular introducer wall defining an introducer lumen formed therethrough. The introducer wall has a proximal portion extending to a distal portion along a longitudinal axis of the introducer sheath. The proximal portion includes a proximal end and the distal portion includes a distal end. A plurality of tubular ports are positioned proximate the proximal end of the introducer sheath. Each tubular port includes a tubular port wall defining a port lumen formed therethrough. Each port lumen is in fluid communication with the introducer lumen and each port wall includes a proximal end extending to a distal end along a central axis of the port. A delivery member is disposed through the proximal end of one of the tubular ports and extends through the port lumen and into the introducer lumen proximate the proximal end of the introducer sheath.

The tubular ports may be formed unitarily with the introducer sheath to define a unitary introducer in which the tubular port walls are formed unitarily with the introducer wall. In one embodiment, the tubular ports may extend radially from the proximal portion of the introducer sheath, wherein the distal end of each tubular port wall extends from the introducer wall at a position distal to the proximal end of the introducer sheath. In another embodiment, the tubular ports may extend from the proximal end of the introducer sheath such that the proximal end of the introducer sheath terminates with the tubular ports.

Alternatively, the tubular ports may be part of an end member separate from and attachable to the proximal end of the introducer sheath, wherein the end member includes a tubular end member wall defining an end member lumen formed therethrough. The end member wall has a proximal end and a distal end. The tubular ports are formed unitarily with the end member to define a unitary end member in which the port walls are formed unitarily with the end member wall. The distal end of the end member is received within the proximal end of the introducer sheath to place the end member lumen and the port lumens in fluid communication with the introducer lumen. In one embodiment, the tubular ports may extend radially from the end member such that the distal end of each tubular port wall extends from the end member wall at a position distal to the proximal end of the end member. In another embodiment, the tubular port walls of the tubular ports may extend from the proximal end of the end member such that the proximal end of the end member terminates with the tubular ports.

In another embodiment of the present invention, a method of delivering multiple delivery members within a patient through a single access site is disclosed. The method includes inserting a multiple port introducer through the access site into a first body lumen of the patient. The multiple port introducer includes an introducer sheath and a plurality of tubular ports positioned proximate a proximal end of the introducer sheath. The introducer sheath has a tubular introducer wall defining an introducer lumen formed therethrough. The introducer wall has a proximal portion extending to a distal portion along a longitudinal axis of the introducer sheath. The proximal portion includes the proximal end and the distal portion includes a distal end. Each tubular port of the plurality of tubular ports includes a tubular port wall defining a port lumen formed therethrough. Each port lumen is in fluid communication with the introducer lumen. Each tubular port wall includes a proximal end extending to a distal end along a central axis of the tubular port.

The method further includes inserting a first delivery member into the proximal end of a first port of the plurality of ports and inserting a second delivery member into the proximal end of a second port of the plurality of ports such that the first and second delivery members extend through the respective port lumens and through the introducer lumen of the introducer sheath into the patient.

Inserting the multiple port introducer into the first body lumen may include positioning the introducer sheath within the first body lumen such that the distal end of the introducer sheath is positioned adjacent at least two branching body lumens including a second body lumen and a third body lumen, each of which branches from the first body lumen, wherein the first delivery member enters the second body lumen and the second delivery member enters the third body lumen as the first and second delivery members exit the distal end of the introducer sheath.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a side plan view of a multiple port introducer in accordance with one embodiment of the present invention, shown without delivery members inserted through the introducer;

FIG. 2 is a side plan view of the multiple port introducer of FIG. 1, shown with delivery members inserted through the introducer;

FIG. 2a is cross-sectional view of the multiple port introducer of FIG. 2, taken along line 2a-2a;

FIG. 3 is a side plan view of a multiple port introducer in accordance with another embodiment of the present invention, shown without delivery members inserted through the introducer;

FIG. 4 is a side plan view of the multiple port introducer of FIG. 3, shown with delivery members inserted through the introducer;

FIG. 4a is cross-sectional view of the multiple port introducer of FIG. 4, taken along line 4a-4a;

FIG. 5 is a side plan view of a multiple port introducer in accordance with another embodiment of the present invention, shown without delivery members inserted through the introducer;

FIGS. 5a-d are cross-sectional views of the multiple port introducer of FIG. 5, taken along respective lines 5a-5a, 5b-5b, 5c-5c, and 5d-5d;

FIG. 6 is a side plan view of a multiple port introducer in accordance with another embodiment of the present invention, shown without delivery members inserted through the introducer; and

FIGS. 6a-d are cross-sectional views of the multiple port introducer of FIG. 6, taken along respective lines 6a-6a, 6b-6b, 6c-6c, and 6d-6d.

### DETAILED DESCRIPTION

Embodiments of the present invention provide a multiple port infusion introducer for use in applications including but not limited to multiple pulmonary arterial embolus thrombolysis, iliac and inferior vena cava thrombolysis, inferior vena cava with branch thrombosis thrombolysis, superior vena cava with branch thrombosis thrombolysis, portal vein and branch thrombosis thrombolysis, low profile multiple segmental branch renal angioplasty, low profile multiple arterial branch thrombosis thrombolysis, and the introduction of multiple wires or snares for foreign body retrieval.

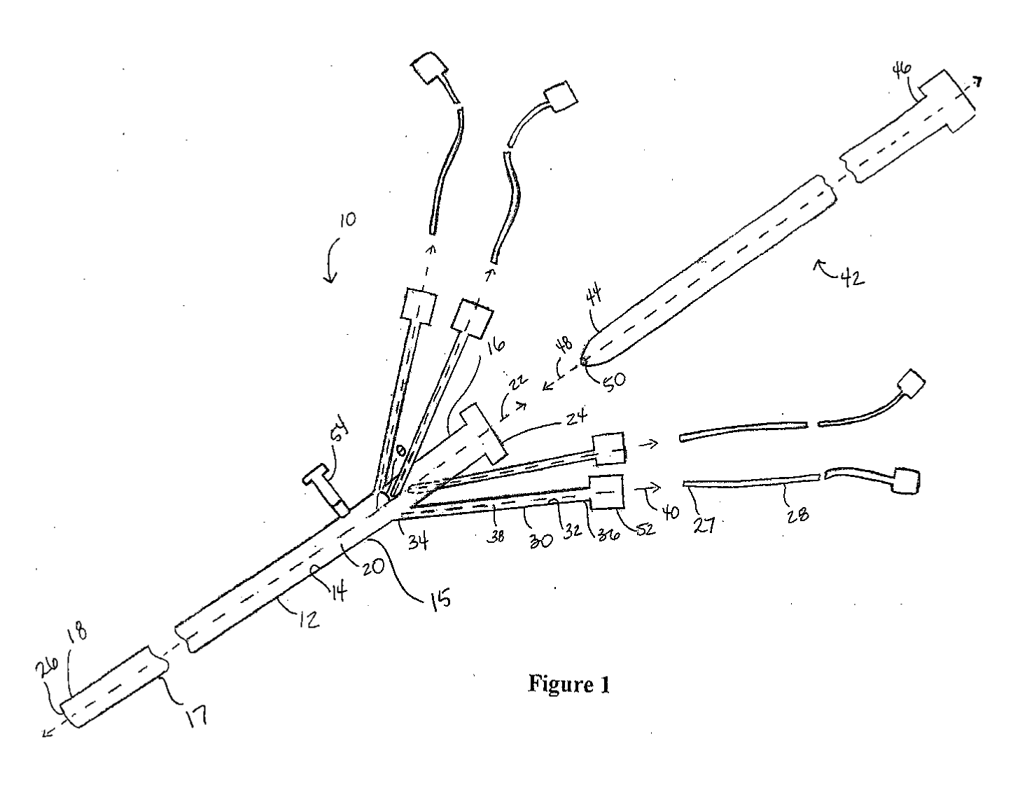

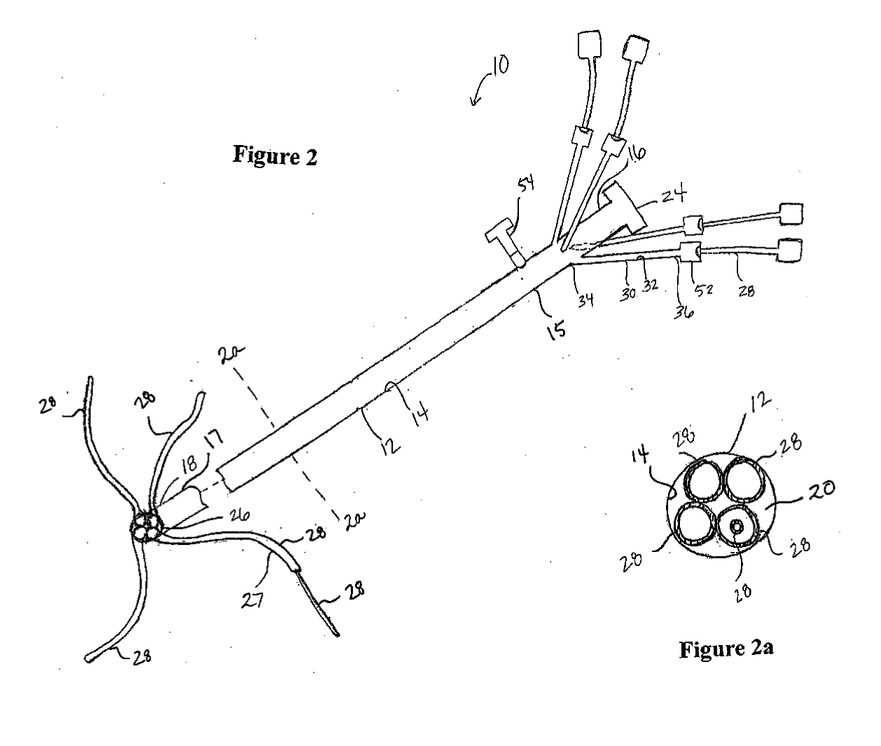

In accordance with a first embodiment of the present invention, FIGS. 1-2a depict a multiple port infusion introducer 10. The introducer 10 includes an outer introducer sheath 12 having a tubular wall 14 including a proximal portion 15 extending to a distal portion 17 along a longitudinal axis 22. The proximal portion 15 includes a proximal end 16 and the distal portion 17 includes a distal end 18. The distal end 18 is configured to be positioned at a location within a patient while the proximal end 16 extends externally from the patient. The tubular wall 14 of the introducer sheath 12 defines a single introducer lumen 20 that extends along the longitudinal axis 22 between a proximal port or opening 24 at the proximal end 16 and a distal port or opening 26 at the distal end 18. The introducer lumen 20 of the introducer sheath 12 is configured to receive a plurality of delivery members 28 at the proximal portion 15, proximate the proximal end 16.

In this embodiment, the introducer sheath 12 includes a plurality of tubular ports 30 formed integrally, i.e., unitarily, with the tubular wall 14 of the introducer sheath 12 and extending radially therefrom to define a unitary introducer 10. As shown in FIG. 1, the tubular ports 30 are arranged about the introducer sheath 12 in a circumferentially staggered arrangement, i.e., the locations or junctions at which the distal ends 34 of the tubular ports 30 extend from the introducer sheath 12 are varied both radially and longitudinally along the tubular wall 14 of the introducer sheath 12.

In this embodiment, each of the tubular ports 30 includes a tubular wall 32 having a distal end 34 formed integrally, i.e., unitarily, with the tubular wall 14 of the introducer sheath 12. The tubular wall 32 extends from the distal end 34 to a proximal end 36 along a longitudinal or central axis 40. As shown in FIG. 1, the tubular wall 32 of each tubular port 30 defines a lumen 38 which extends along the central axis 40 of the tubular port 30. Each of the tubular ports 30 extends radially from the introducer sheath 12 such that the central axis 40 of each tubular port 30 intersects the longitudinal axis 22 of the introducer sheath 12 at an angle θ, wherein angle θ is preferably between about 20° and about 60°.

As described in further detail below, each of the tubular ports 30 is configured to receive a delivery member 28. A distal end 27 of the delivery member 28 enters the tubular port 30 via the proximal end 36. The delivery member 28 travels through the lumen 38 of the tubular port 30 and into the lumen 20 of the introducer sheath 12 at the proximal portion 15 of the introducer sheath 12. The delivery members 28 extend simultaneously through the introducer lumen 20 and exit the distal opening 26 at the distal end 18 of the introducer sheath 12 to enter a desired delivery site within a patient. Preferably, the delivery members 28 include tubular delivery members, such as catheters or infusion wires for the delivery of therapeutic agents to the desired delivery site within the patient. Other delivery members 28 include guidewires, snares, balloons and the like for various medical applications including but not limited to drug delivery, foreign body retrieval, and angioplasties.

A method of using the multiple port introducer 10 preferably includes inserting the introducer 10 within a body lumen of the patient, wherein the tubular ports 30 extending radially from the proximal portion 15 of the introducer sheath 12 are accessible outside of the patient's body to thereafter insert a plurality of delivery members 28 within the patient to deliver a therapeutic agent or drug to the desired delivery site within the patient, or to perform other various medical procedures.

Inserting the introducer 10 preferably includes navigating a guidewire through the vasculature of the patient to an appropriate delivery site. Thereafter, the introducer 10 receives an introducer dilator 42 for inserting the introducer 10 into the patient. The introducer dilator 42 may be any dilator known to one of skill in the art used in similar medical applications. As shown in FIG. 1, the introducer dilator 42 includes a distal end 44 extending to a proximal end 46 along a longitudinal axis 48. The introducer dilator 42 enters the introducer lumen 20 of the introducer sheath 12 through the proximal opening 24 at the proximal end 16, wherein the longitudinal axis 48 of the introducer dilator 42 coincides with the longitudinal axis 22 of the introducer sheath 12. The introducer dilator 42 includes an opening 50 at the distal end 44 which receives the guidewire for guiding the introducer dilator 42, and thus the introducer sheath 12, through the patient's body to the appropriate delivery site. The distal end 44 of the introducer dilator 42 is tapered to aid insertion and advancement of the introducer sheath 12 into the patient's body. The distal end 18 of the introducer sheath 12 may include a radiopaque marker or other diagnostic device to aid in positioning of the introducer 10.

Once the introducer 10 is properly positioned within the patient's body, the guidewire and the introducer dilator 42 are removed from within the introducer sheath 12 and from within the patient. Thereafter, delivery members 28 including but not limited to inner catheters, infusion wires, infusion wires disposed coaxially within inner catheters, snares, or balloons are inserted into the introducer sheath 12 though the tubular ports 30, as illustrated in FIG. 2. Preferably, the delivery members 28 are tubular delivery members configured to receive a guidewire, wherein the guidewires are inserted through the tubular ports 30 and used to aid in the insertion of the delivery members 28 so that the delivery members 28 do not become entangled within the single lumen 20 of the introducer sheath 12. FIG. 2a illustrates a plurality of delivery members 28 disposed within the single lumen 20 of the introducer sheath 12 along a central portion of the introducer sheath. In this embodiment, distal ends 27 of the delivery members 28 are inserted over the guidewires at the proximal ends 36 of the tubular ports 30 and advanced through the tubular ports 30 and into the introducer sheath 12 and further advanced through the introducer sheath 12 toward the distal end 18 of the introducer sheath 12. As shown in FIG. 2, the delivery members 28 exit the distal opening 26 at the distal end 18 of the introducer sheath 12 and preferably branch into separate veins or arteries for the delivery of fluid agents. Once the delivery members 28 have reached the desired delivery site within the patient's body, the guidewires are removed and the therapeutic agent or drug is flushed through the delivery members 28 and into the desired veins or arteries within the patient.

In this embodiment, each of the lumens 38 of the tubular ports 30 is provided with a suitable conventional hemostatic valve 52 which allows sealable introduction of the delivery members 28 into the tubular ports 30. Similarly, one or more seals or valves (e.g., a hemostatic valve, not shown) may be provided on the proximal end 16 of the introducer sheath to seal the lumen 20, and thus prevent proximal flow, while allowing devices, e.g., delivery members 28, as well as the introducer dilator 42, to be introduced into the lumen 20. In addition, a side flush port 54 is preferably connected to the introducer sheath 12 at the proximal portion 15 for flushing a solution of heparin, saline, glucose solution and/or the like within the introducer sheath 12 around the delivery members 28 to prevent clotting of blood flowing therethrough. After delivery of the fluid agent to the veins or arteries, or the performance of any other suitable medical procedure, the introducer 10 is removed from within the patient's body.

Preferably, the introducer sheath 12 is sized between about 6 Fr and about 12 Fr. The introducer sheath 12 is preferably about 30 cm in length to about 70 cm in length. Each of the delivery members 28 is preferably sized up to about 6 Fr. While the above embodiments include four tubular ports 30, this is merely an example of a number of multiple ports the introducer 10 may include and thus the introducer 10 may include two, three, four, or more than four tubular ports 30 as long as the plurality of delivery members 28 received within the tubular ports 30 and advanced to the introducer sheath 12 fits within the lumen 20 of the introducer sheath 12. Likewise, the number of delivery members 28 may be varied so long as the lumen 20 of the introducer sheath 12 is sufficient to receive the number of delivery members 28.

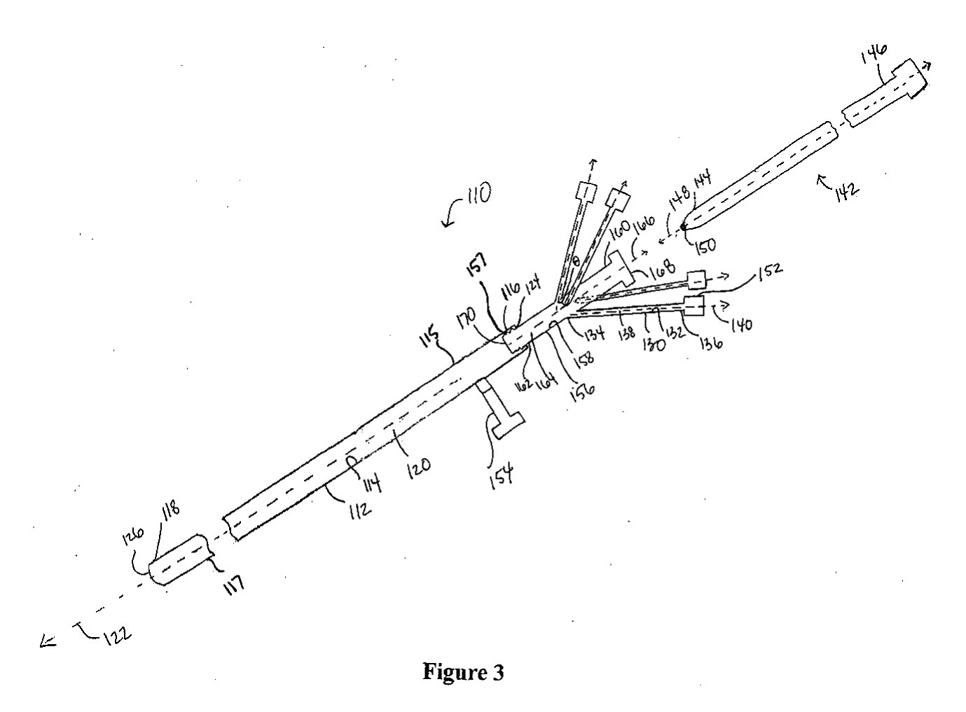

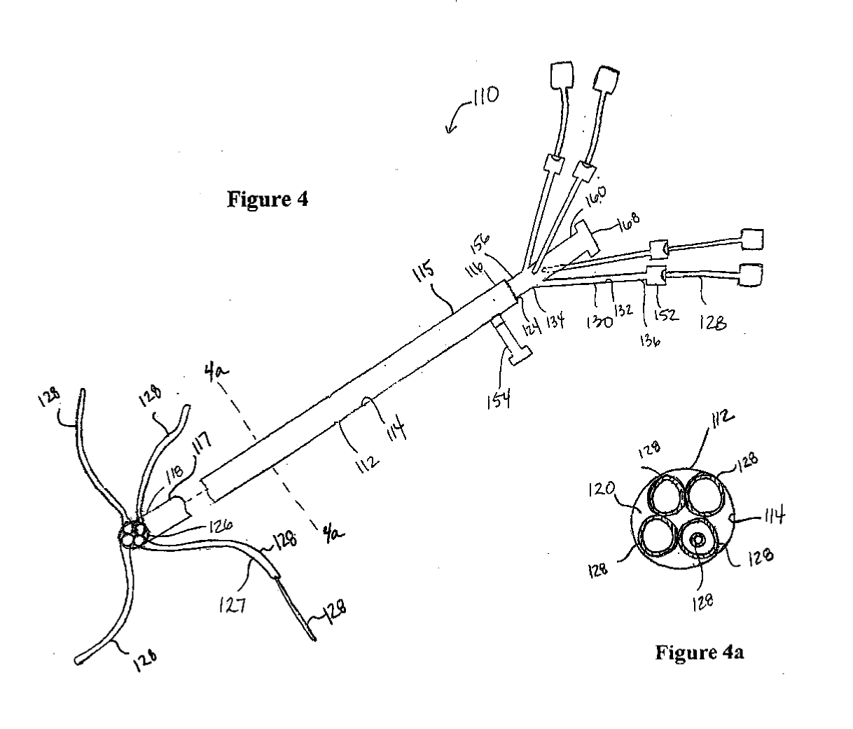

FIGS. 3-4a depict a second embodiment of a multiple port thrombolysis infusion introducer 110 in accordance with the teachings of the present invention and having a description similar to that of FIGS. 1-2a, and in which similar components are denoted by similar reference numerals increased by 100. In this embodiment, the multiple port introducer 110 includes an outer introducer sheath 112 having a tubular wall 114 including a proximal portion 115 extending to a distal portion 117 along a longitudinal axis 122. The proximal portion 115 includes a proximal end 116 and the distal portion 117 includes a distal end 118. The distal end 118 is configured to be positioned at a location within a patient while the proximal end 116 extends externally from the patient. The tubular wall 114 of the introducer sheath 112 defines a single introducer lumen 120 that extends along the longitudinal axis 122 between a proximal port or opening 124 at the proximal end 116 and a distal port or opening 126 at the distal end 118. The introducer lumen 120 of the outer introducer sheath 112 is configured to receive a plurality of delivery members 128 proximate the proximal end 116.

In this embodiment, the proximal opening 124 of the introducer sheath 112 receives a separate or modular tubular end member 156 having a plurality of tubular ports 130, i.e., the tubular end member 156 is separate from and attachable to the introducer sheath 112 at the proximal opening 124 thereof. The tubular end member 156 includes a tubular wall 158 having a proximal end 160 and a distal end 162. The tubular wall 158 of the tubular end member 156 defines a lumen 164 that extends along a longitudinal axis 166 between a proximal port or opening 168 at the proximal end 160 and a distal port or opening 170 at the distal end 162. In this embodiment, the plurality of tubular ports 130 is formed integrally, i.e., unitarily, with the tubular wall 158 of the tubular end member 156 to define a unitary end member. As illustrated in FIG. 3, the tubular ports 130 extend from the tubular end member 156 proximate the proximal end 160 in a circumferentially staggered arrangement, i.e., the locations or junctions at which the distal ends 134 of the tubular ports 130 extend from the tubular end member 156 are varied both radially and longitudinally along the tubular wall 158 of the tubular end member 156.

As illustrated in FIGS. 3-4, each of the tubular ports 130 includes a tubular wall 132 having a distal end 134 formed integrally as one with the tubular wall 158 of the tubular end member 156. The tubular wall 132 extends from the distal end 134 to a proximal end 136 along a longitudinal or central axis 140. As shown in FIG. 3, the tubular wall 132 of each tubular port 130 defines a lumen 138 which extends along the central axis 140 of the tubular port 130. Each of the tubular ports 130 extends radially from the tubular end member 156 such that the central axis 140 of each tubular port 130 intersects the longitudinal axis 166 of the tubular end member 156, as well as the longitudinal axis 122 of the introducer sheath 112, at an angle θ, wherein angle θ is preferably between about 20° and 60°.

As described in further detail below, each of the tubular ports 130 is configured to receive a delivery member 128. A distal end 127 of the delivery member 128 enters the tubular port 130 via the proximal end 136. The delivery member 128 travels through the lumen 138 of the tubular port 130 and into the lumen 164 of the tubular end member 156 and further into the lumen 120 of the introducer sheath 112 at the proximal portion 15 thereof. The delivery members 128 extend simultaneously through the lumen 120 of the introducer sheath 112 and exit the distal opening 126 thereof to enter a desired delivery site within a patient. The delivery members 128 may include any of those disclosed above with respect to FIGS. 1-2a.

A method of using the multiple port introducer 110 preferably includes inserting the introducer 110 within a body lumen of the patient, wherein the radially extending tubular ports 130 positioned proximate the proximal end 116 of the introducer 10 are accessible outside of the patient's body to thereafter insert a plurality of delivery members within the patient to deliver a therapeutic agent or drug to the desired delivery site within the patient, or to perform other various medical procedures.

Inserting the introducer 10 preferably includes navigating a guidewire through the vasculature of the patient to an appropriate delivery site. Thereafter, the introducer 110 receives an introducer dilator 142 for inserting the introducer 110 into the patient. In this embodiment, the introducer dilator 142 includes a distal end 144 extending to a proximal end 146 along a longitudinal axis 148. The introducer dilator 142 includes an opening 150 at the distal end 144 which receives the guidewire for guiding the introducer dilator 142, and thus the introducer sheath 112, through the patient's body to the appropriate delivery site. The distal end 144 of the introducer dilator 142 is tapered to aid insertion and advancement of the introducer sheath 112 into the patient's body. The distal end 118 of the introducer sheath 12 may include a radiopaque marker or other diagnostic device to aid in positioning of the introducer 110.

In this embodiment, the introducer dilator 142 is preferably directly received within the introducer sheath 112 to guide the introducer sheath 112 to the delivery site prior to assembly of the tubular end member 156 with the introducer sheath 12. In this embodiment, the introducer dilator 142 is received within the introducer sheath 112 via the proximal opening 124 to advance the introducer sheath 112 through the patient's body to the delivery site. Once the introducer sheath 112 is positioned appropriately within the patient's body, the guidewire and the introducer dilator 142 are removed. Thereafter, the tubular end member 156 is assembled with the introducer sheath 112. As shown in FIGS. 3-4, the tubular end member 156 is received within the lumen 120 of the introducer sheath 112 via the proximal opening 124 of the introducer sheath 112. The tubular end member 156 preferably includes an introducer sheath fit member 157 which couples the tubular end member 156 to the proximal end 116 of the introducer sheath 112. The tubular end member 156 may be assembled with the introducer sheath 112 by any suitable means know by one of skill in the art including, for example, a screw fit with corresponding male and female threads.

Alternatively, the tubular end member 156 may be pre-assembled with the introducer sheath 12 prior to insertion of the introducer 110 aided by the introducer dilator 142. In this example, the tubular end member 156 is received within the lumen 120 of the introducer sheath 112 via the proximal opening 124, wherein the longitudinal axis 166 of the tubular end member 156 coincides with the longitudinal axis 122 of the introducer sheath. In this embodiment, the introducer dilator 142 enters the lumen 164 of the tubular end member 156 via the proximal opening 168, wherein the longitudinal axis 148 of the introducer dilator 142 coincides with the longitudinal axes 166, 122 of the tubular end member 156 and the introducer sheath 112, respectively. The introducer dilator 142 advances into the lumen 120 of the introducer sheath 112 via the distal opening 170 of the tubular end member 156 and the proximal opening 124 of the introducer sheath 112. Once the introducer 110 is positioned appropriately within the patient's body, the guidewire and the introducer dilator 142 are removed from within the introducer sheath 112 and the tubular end member 156.

Thus, in the second embodiment, the introducer dilator 142 may be used to position the introducer sheath 112 within the patient either before or after the tubular end member 156 is connected to the introducer sheath 112. After the positioning of the introducer sheath 112, and the subsequent removal of the introducer dilator 142, the tubular ports 130 receive a plurality of delivery members 128. FIG. 4a illustrates a plurality of delivery members 128 disposed within the single lumen 120 of the introducer sheath 112 along a central portion of the introducer sheath.

Preferably, distal ends 127 of the delivery members 128 are inserted over guidewires at the proximal ends 136 of the tubular ports 130 and advanced through the tubular ports 130 into the tubular end member 156 and into the introducer sheath 112 through the distal opening 170 of the tubular end member 156. The delivery members 128 are further advanced through the introducer sheath 112 toward the distal end 118 of the introducer sheath 112. As shown in FIG. 4, the delivery members 128 exit the distal opening 126 of the introducer sheath 112 and preferably branch into separate veins or arteries for the delivery of fluid agents. Once the delivery members 128 have reached the desired delivery site within the patient's body, the guidewires are removed and the therapeutic agent or drug is flushed through the delivery members 128 and into the desired veins or arteries within the patient.

In this embodiment, each of the lumens 138 of the tubular ports 130 is provided with a suitable conventional hemostatic valve 152 which allows sealable introduction of the delivery members 128 into the tubular ports 130. Similarly, one or more seals or valves (e.g., a hemostatic valve, not shown) may be provided on the proximal end 160 of the tubular end member 156 and the proximal end 116 of the introducer sheath 112 to seal respective lumens 164, 120, and thus prevent proximal flow, while allowing devices, e.g., tubular delivery devices 128, such as guidewires, as well as the introducer dilator 142, to be introduced into the lumens 164, 120. In addition, a side flush port 154 is preferably connected to the introducer sheath 112 at the proximal portion 115 for flushing a solution of heparin, saline, glucose solution and/or the like within the introducer sheath 112 around the delivery members 128 to prevent clotting of blood flowing therethrough. After delivery of the fluid agent to the veins or arteries, the introducer 110 is removed from the patient's body.

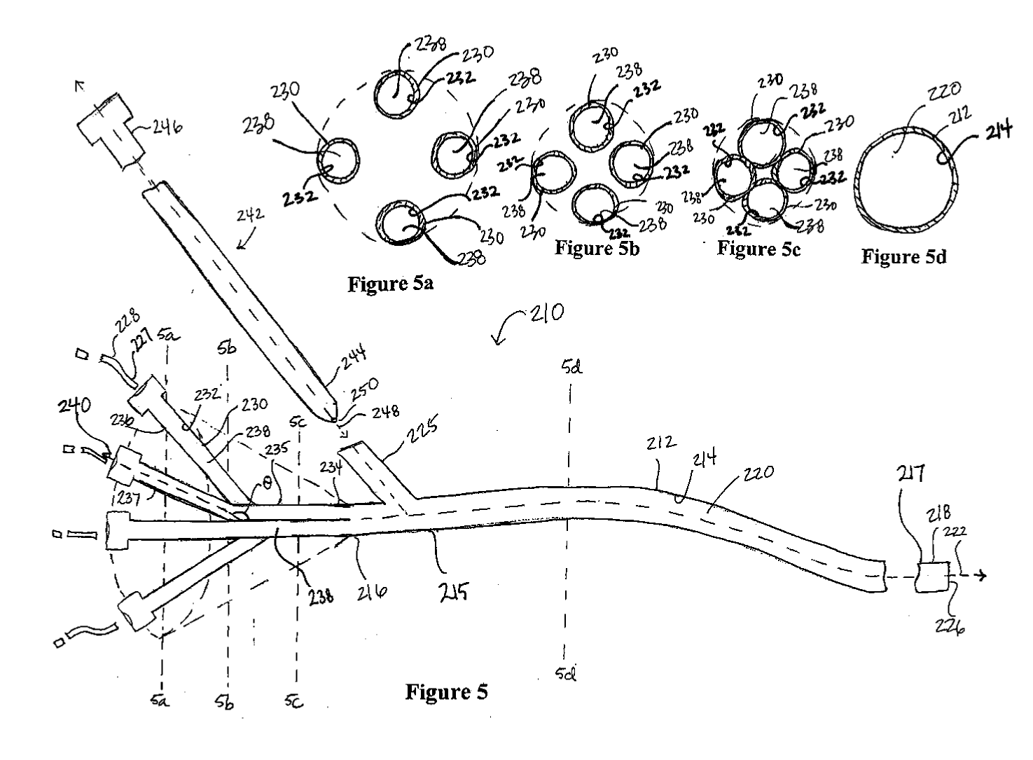

FIGS. 5 and 5a-d depict a third embodiment of a multiple port thrombolysis infusion introducer 210 in accordance with the teachings of the present invention and having a description similar to that of FIGS. 1-2a, and in which similar components are denoted by similar reference numerals increased by 200. The multiple port introducer 210 includes an outer introducer sheath 212 having a tubular wall 214 including a proximal portion 215 extending to a distal portion 217 along a longitudinal axis 222. The proximal portion 215 includes a proximal end 216 and the distal portion 217 includes a distal end 218. The distal end 218 is configured to be positioned at a location within a patient while the proximal end 216 extends externally from the patient. The tubular wall 214 of the introducer sheath 212 defines a single introducer lumen 220 that extends along the longitudinal axis 222 between the proximal and distal ends 216, 218. The introducer lumen 220 of the outer introducer sheath 212 is configured to receive a plurality of delivery members 228 at the proximal portion 215, proximate the proximal end 216.

In this embodiment, the introducer sheath 212 includes a plurality of tubular ports 230 formed integrally, i.e., unitarily, with the tubular wall 214 of the introducer sheath 212 and extending radially therefrom to define a unitary introducer 210. As shown in FIG. 5, the tubular ports 230 extend from the proximal end 216 of the introducer sheath 210, defining a plurality of respective lumens 238 which meet the single introducer lumen 220 at the proximal end 216 of the introducer sheath 212. In other words, the proximal end 216 of the introducer sheath 212 terminates with the plurality of tubular ports 230. Each of the tubular ports 230 includes a tubular wall 232 having a distal end 234 formed integrally, i.e., unitarily, with the introducer sheath 212 and extending to a proximal end 236. The tubular wall 232 of each tubular port 230 defines a lumen 238. Each of the tubular ports 230 includes a first portion 235 which extends longitudinally from the introducer sheath 212 such that a central axis defined by the first portion 235 of each tubular port 230 is parallel to the longitudinal axis 222 of the introducer sheath 21, and a second portion 237 which extends radially from the first portion 235 such that a central axis 240 defined by the second portion 237 of each tubular port 230 intersects the longitudinal axis 222 of the introducer sheath 212 at an angle θ, wherein angle θ is preferably between about 20° and 60°.

As described in further detail below, each of the tubular ports 230 is configured to receive a delivery member 228. A distal end 227 of the delivery member 228 enters the tubular port 230 via the proximal end 236. The delivery member 228 travels through the lumen 238 of the tubular port 230 and into the lumen 220 of the introducer sheath 212 at the proximal portion 215 of the introducer sheath 12. The delivery members 228 extend simultaneously through the lumen 220 of the introducer sheath 212 and exit the distal opening 226 thereof to enter a desire delivery site within a patient. The delivery members 228 may include any of those disclosed above with respect to FIGS. 1-2a.

A method of using the multiple port introducer 210 preferably includes inserting the introducer 210 within the patient's body, wherein the tubular ports 230 extending radially from the proximal end 216 of the introducer sheath 12 are accessible outside of the patient's body to thereafter insert a plurality of delivery members 228 within the patient to deliver a therapeutic agent or drug to the desired delivery site within the patient, or to perform other various medical procedures.

Inserting the introducer 210 preferably includes navigating a guidewire through the vasculature of the patient to an appropriate delivery site. Thereafter, the introducer sheath 212 receives an introducer dilator 242 for inserting the introducer 10 into the patient. As shown in FIG. 5, an introducer dilator port 225 formed integrally, i.e., unitarily, with the tubular wall 214 extends from the introducer sheath 212 at an angle and is disposed distally relative to the proximal end 216 of the introducer sheath 212. The introducer dilator port 225 is configured to receive the introducer dilator 242 for positioning the introducer sheath 212 within the patient. After positioning of the introducer sheath 212, and the subsequent removal of the introducer dilator 242, the tubular ports 230 receive a plurality of delivery members 228 including but not limited to inner catheters, infusion wires, infusion wires coaxially disposed within inner catheters, snares, balloons, diagnostic catheters, or the like.

In this embodiment, each of the lumens 238 of the tubular ports 230 is provided with a suitable conventional hemostatic valve 252 which allows sealable introduction of the delivery members 228 into the tubular ports 230. The delivery members 228 pass through the lumens 238 of the tubular ports 230 and enter the single lumen 220 of the introducer sheath 212 at the proximal end 216 (i.e., where the distal ends 234 of the tubular ports 230 meet the proximal end 216 of the introducer sheath 212) and are advanced through the lumen 220 out the distal opening 226 of the introducer sheath 212 for delivery of a therapeutic agent to the desired delivery site. A side flush port may be connected to the introducer sheath 312 at the proximal portion 215 for flushing a solution of heparin, saline, glucose solution and/or the like within the introducer sheath 312 around the delivery members 328 to prevent clotting.

As illustrated in FIGS. 5a-d, the lumens 238 of the tubular ports 230 are arranged in a funnel-like configuration, being widely spaced from one another along line 5a-5a and converging closer together in lines 5b-5b and 5c-5c until they meet the proximal end 216, and the single introducer lumen 220, of the introducer sheath 212. Preferably, this funnel-like configuration decreases the risk of the delivery members 238 entangling once within the single lumen 220 of the introducer sheath 212.

It is also within the scope of the present invention for the tubular ports 230 to be part of a separate tubular end member attachable to the introducer sheath 212 at the proximal end 216 thereof in similar fashion as the embodiment disclosed in FIG. 3.

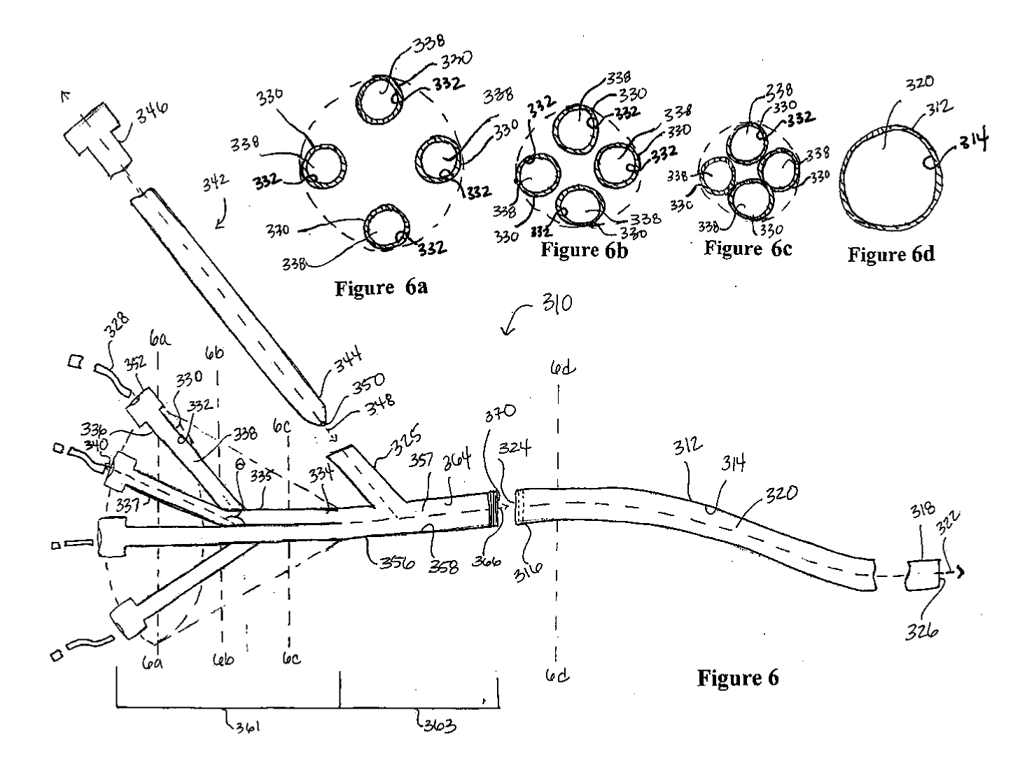

FIGS. 6 and 6a-d depict a fourth embodiment of a multiple port thrombolysis infusion introducer 310 in accordance with the teachings of the present invention and having a description similar to that of FIGS. 5 and 5a-d, and in which similar components are denoted by similar reference numerals increased by 100. This embodiment is similar to the third embodiment depicted in FIGS. 5 and 5a-d, however, the tubular ports 330 are part of a separate or modular tubular end member 356 configured to connect to the introducer sheath 312 i.e., the tubular end member 356 is separate from and attachable to the introducer sheath 312 at the proximal opening 324 thereof. In this embodiment, the introducer sheath 312 includes a tubular wall 314 including a proximal portion 315 extending to a distal portion 317 along a longitudinal axis 322. The proximal portion 315 includes a proximal end 316 and the distal portion includes a distal end 318. The distal end 318 is configured to be positioned at a location within a patient while the proximal end 316 is extends externally from the patient. The tubular wall 314 of the introducer sheath 312 defines a single introducer lumen 320 that extends along a longitudinal axis 322 between a proximal port or opening 324 at the proximal end 316 and a distal port or opening 326 at the distal end 318. The introducer lumen 320 of the introducer sheath 312 is configured to receive a plurality of delivery members 328 at the proximal end 316.

As shown in FIG. 6, the tubular ports 330 are formed within a proximal portion 361 of the tubular end member 356. The plurality of tubular ports 330 includes a plurality of respective lumens 338 within the proximal portion 361 of the tubular end member 356, each of which extends distally to a single lumen 357 defined within the distal portion 363 of the tubular end member 356. Each of the tubular ports 330 includes a first portion 335 which extends longitudinally from the distal portion 363 of the tubular end member 356 such that a central axis defined by the first portion 335 of each tubular port 330 is parallel to the longitudinal axis 366 defined by the tubular end member 356, and a second portion 337 which extends radially from the first portion 335 such that a central axis 340 defined by the second portion 337 of each tubular port 330 intersects the longitudinal axis 366 defined by the tubular end member 356 at an angle θ, wherein angle θ is preferably between about 20° and 60°.

In this embodiment, each of the lumens 338 of the tubular ports 330 is provided with a suitable conventional hemostatic valve 352 which allows sealable introduction of the delivery members 328 into the tubular ports 330. In addition, a side flush port may be connected to the introducer sheath 312 at the proximal portion 315 for flushing a solution of heparin, saline, glucose solution and/or the like within the introducer sheath 312 around the delivery members 328 to prevent clotting.

In this embodiment, the tubular end member 356 connects to the introducer sheath 312 by any suitable means known in the art, such as through welding or a screw-fit. When the tubular end member 356 is connected to the introducer sheath 312, the longitudinal axes 366 and 322 align such that the distal opening 370 of the tubular end member 356 meets the proximal opening 324 of the introducer sheath 312.

Referring to FIG. 6, the tubular end member 356 includes an introducer dilator port 325 configured to receive an introducer dilator 342 for positioning the introducer sheath 312 within the patient. After the positioning of the introducer sheath 312 and the subsequent removal of the introducer dilator 342, each of the tubular ports 330 receives a delivery member 328 including but not limited to inner catheters, infusion wires, infusion wires coaxially disposed within inner catheters, snares, balloons, diagnostic catheters, or the like. In this embodiment, the introducer dilator 342 extends from the distal portion 363 of the tubular end member 356 at an angle so that as the delivery members 328 enter the single lumen 357 within the distal portion 363 of the tubular end member 356, the delivery members 328 do not select the path of the dilator port 325. Rather, the delivery members 328 advance toward the introducer lumen 320 and out the distal port 326 of the introducer sheath 312 for delivery of a therapeutic agent to the desired delivery site.

As illustrated in FIGS. 6a-d, the lumens 338 of the tubular ports 330 are arranged in a funnel-like configuration, being widely spaced from one another along line 6a-6a and converging closer together in lines 6b-6b and 6c-6c until they meet the single lumen 357 of the distal portion 363 of the tubular end member 356. Preferably, this funnel-like configuration decreases the risk of the delivery members 338 entangling once within the single lumen 320 of the introducer sheath 312.

It should be apparent to those skilled in the art that the above-described embodiments are merely illustrative of but a few of the many possible specific embodiments of the present invention. Numerous and various other arrangements can be readily devised by those skilled in the art without departing from the spirit and scope of the invention as defined in the following claims.

1. A multiple port introducer comprising:
an introducer sheath having a tubular introducer wall defining an introducer lumen formed therethrough, the introducer wall having a proximal portion extending to a distal portion along a longitudinal axis of the introducer sheath, the proximal portion including a proximal end and the distal portion including a distal end;
a plurality of ports positioned proximate the proximal end of the introducer sheath, each port having a tubular port wall defining a port lumen formed therethrough, wherein each port lumen is in fluid communication with the introducer lumen, each port wall having a proximal end extending to a distal end along a central axis of the port; and
a delivery member disposed through the proximal end of one of the ports, the delivery member extending through the port lumen and into the introducer lumen proximate the proximal end of the introducer sheath.

2. The introducer of claim 1, wherein the ports are formed unitarily with the introducer sheath to define a unitary introducer, the port walls being formed unitarily with the introducer wall.

3. The introducer of claim 2, wherein the ports extend radially from the proximal portion of the introducer sheath, the distal end of each port wall extending from the introducer wall at a position distal to the proximal end of the introducer sheath.

4. The introducer of claim 3, wherein the ports extend radially from the introducer sheath such that the central axis of each port intersects the longitudinal axis of the introducer sheath at an angle θ, wherein angle θ is between about 20 degrees and about 60 degrees.

5. The introducer of claim 3, wherein the ports extend radially from the introducer sheath in a circumferentially staggered arrangement.

6. The introducer of claim 2, wherein the port walls of the ports extend from the proximal end of the introducer sheath such that the proximal end of the introducer sheath terminates with the ports.

7. The introducer of claim 6, wherein each port includes a proximal portion and a distal portion, wherein the central axis of each port along the distal portion thereof is parallel to the longitudinal axis of the introducer sheath, and wherein the central axis of each port along the proximal portion thereof intersects the longitudinal axis of the introducer sheath at an angle θ.

8. The introducer of claim 1, wherein the ports are part of an end member separate from and attachable to the proximal end of the introducer sheath, the end member having a tubular end member wall defining an end member lumen formed therethrough, the end member wall having a proximal end and a distal end, wherein the ports are formed unitarily with the end member to define a unitary end member, the port walls being formed unitarily with the end member wall, wherein the distal end of the end member is received within the proximal end of the introducer sheath to place the end member lumen and the port lumens in fluid communication with the introducer lumen.

9. The introducer of claim 8, wherein the ports extend radially from the end member, the distal end of each port wall extending from the end member wall at a position distal to the proximal end of the end member.

10. The introducer of claim 9, wherein the ports extend radially from the tubular end member in a circumferentially staggered arrangement, the end member positioned within the introducer sheath such that the central axis of each port intersects the longitudinal axis of the introducer sheath at an angle θ.

11. The introducer of claim 8, wherein the port walls of the ports extend from the proximal end of the end member such that the proximal end of the end member terminates with the ports.

12. The introducer of claim 11, wherein each port includes a proximal portion and a distal portion, wherein the end member is positioned within the introducer sheath such that the central axis of each port along the distal portion thereof is parallel to the longitudinal axis of the introducer sheath and such that the central axis of each port along the proximal portion thereof intersects the longitudinal axis of the introducer sheath at an angle θ.

13. The introducer of claim 1, wherein the proximal end of the introducer sheath is configured to receive an introducer dilator for positioning the introducer sheath within a patient.

14. The introducer of claim 1, wherein the proximal portion of the introducer sheath includes a dilator port extending at an angle therefrom configured to receive an introducer dilator for positioning the introducer sheath within a patient.

15. The introducer of claim 1, wherein the proximal portion of the introducer sheath includes a fluid flush port attached thereto and in fluid communication with the introducer lumen, wherein the fluid flush port provides a flush solution to the introducer lumen to prevent clotting of blood flowing therethrough.

16. The introducer of claim 1, wherein the delivery member includes at least one of a catheter, infusion wire, guidewire, snare, and a balloon.

17. The introducer of claim 1, wherein the delivery member is one of a plurality of delivery members, each delivery member being disposed through the proximal end of one of the ports, wherein the delivery members extend through the introducer lumen and exit the distal end of the introducer sheath to deliver a fluid agent to a desired delivery site within a patient's body.

18. A method of delivering multiple delivery members within a patient through a single access site, the method comprising:
inserting a multiple port introducer through the access site into a first body lumen of the patient, the multiple port introducer including an introducer sheath and a plurality of ports positioned proximate a proximal end of the introducer sheath, the introducer sheath having a tubular introducer wall defining an introducer lumen formed therethrough, the introducer wall having a proximal portion extending to a distal portion along a longitudinal axis of the introducer sheath, the proximal portion including the proximal end and the distal portion including a distal end, each port of the plurality of ports having a tubular port wall defining a port lumen formed therethrough, wherein each port lumen is in fluid communication with the introducer lumen, each port wall having a proximal end extending to a distal end along a central axis of the port; and
inserting a first delivery member into the proximal end of a first port of the plurality of ports and inserting a second delivery member into the proximal end of a second port of the plurality of ports such that the first and second delivery members extend through the respective port lumens and through the introducer lumen of the introducer sheath into the patient.

19. The method of claim 18, wherein inserting the multiple port introducer into the first body lumen includes positioning the introducer sheath within the first body lumen such that the distal end of the introducer sheath is positioned adjacent at least two branching body lumens including a second body lumen and a third body lumen, each of which branches from the first body lumen, wherein the first delivery member enters the second body lumen and the second delivery member enters the third body lumen.

20. The method of claim 19, further comprising injecting a therapeutic agent into the first and second delivery members to deliver the therapeutic agent to the second and third body lumens.
